(19) United States
(12) Patent Application Publication
(10) Pub. No.: US 2006/0154853 A1
(43) Pub. Date: Jul. 13, 2006

(54) Method of treating an autoimmune disease

(76) Inventors: Raymond John Steptoe, Victoria (AU); Leonard Charles Harrison, Victoria (AU)

Correspondence Address: SCULLY, SCOTT, MURPHY & PRESSER, 400 GARDEN CITY PLAZA, SUITE 300, GARDEN CITY, NY, 11530, US

(21) Appl. No.: 10/527,925

(22) PCT Filed: Sep. 16, 2003
(86) PCT No.: PCT/AU03/01212

§ 371 Date: Sep. 15, 2005

(30) Foreign Application Priority Data
Sep. 16, 2002 (AU) 2002952834

(51) Int. Cl. A61K 38/28

(52) U.S. Cl. 514/3

(57) ABSTRACT

The present invention relates generally to a method for treating or ameliorating the symptoms of or reducing or otherwise minimizing the risk of development of an autoimmune disease such as but not limited to autoimmune diabetes. More particularly, the present invention relates to the use of genetically modified hemopoietic stem cells and/or hemopoietic progenitor cells which express genetic material encoding one or more autoantigens which give rise to antigen-presenting cells that induce immune tolerance and/or protective immunity. The present invention provides, therefore, a method for the treatment and/or prophylaxis of autoimmune disease conditions such as type 1 diabetes.

### BACKGROUND OF THE INVENTION

1. Field of the Invention

The present invention relates generally to a method for treating or ameliorating the symptoms of or reducing or otherwise minimizing the risk of development of an autoimmune disease such as but not limited to autoimmune diabetes. More particularly, the present invention relates to the use of genetically modified hemopoietic stem cells and/or hemopoietic progenitor cells which express genetic material encoding one or more autoantigens which give rise to antigen-presenting cells that induce immune tolerance and/or protective immunity. The present invention provides, therefore, a method for the treatment and/or prophylaxis of autoimmune disease conditions such as type 1 diabetes.

2. Description of the Prior Art

Bibliographic details of references provided in the subject specification are listed at the end of the specification.

Reference to any prior art in this specification is not, and should not be taken as, an acknowledgment or any form of suggestion that this prior art forms part of the common general knowledge in any country.

Insulin-dependent or type 1 diabetes is caused by a lack of insulin, due to autoimmune-mediated destruction of pancreatic islet p cells. Individuals with type 1 diabetes require regular insulin injections to control their blood glucose levels. Failure to treat individuals in this manner can lead to death.

A more long term treatment strategy is required with the prevention of the autoimmune condition being the principal goal.

Pancreas transplantation is currently the only curative therapy for type 1 diabetes, but this is hampered by the requirement for potentially toxic, life-long immunosuppressive drugs and by the dearth of human donors.

Bone marrow (BM) or hematopoietic stem cell (HSC) transplantation has recently been used to treat clinically severe autoimmune disease (Burt et al., Blood 99: 768-784, 2002). In pre-clinical animal models, effective treatment of spontaneous autoimunune disease requires transplantation of BM or HSC from disease-resistant strains. To date, this has been achieved by allogeneic BM transplantation (BMT) (Ikehara et al., Proc. Natl. Acad. Sci. USA 82: 7743-7747, 1985; LaFace and Peck, Diabetes 38: 894-901, 1989; El-Badri et al., Transplantation 70: 870-877, 2000; Himeno and Good, Proc. Natl. Acad. Sci. USA 85: 2235-2239, 1998; Kirzner et al., Biol. Blood Marrow Transplant. 6: 513-522, 2000) leading to full or mixed chimerism (Li et al., J. Immunol. 156: 380-388, 1996; Kaufman et al., J. Immunol. 158: 2435-2442, 1997). However, the requirement for cytotoxic conditioning of the host and the risk of graft rejection (Castro-Malaspina et al., Blood 99: 1943-1951, 2002) or graft-versus-host disease Ratanatharathorn et al., Bone Marrow Transplant 28: 121-129, 2001) render this approach unsuitable for widespread clinical application.

There is a need, therefore, to develop alternative strategies and approaches which prevent the development of autoimmune diabetes as well as other autoimmune disease conditions.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers. Targeting antigen to resting antigen-presenting cells (APCs), such as B cells, dendritic cells, epithelial cells or macrophages amongst others, is proposed as a means of inducing immunological unresponsiveness (tolerance) and/or protective immunity. As all immune cells are derived from hemopoetic stem cells (HSCs) and hemopoietic progenitor cells (HPCs), it is proposed, in accordance with the present invention, that HSCs and/or HPCs encoding an autoantigen will develop into APCs expressing the autoantigen. These are then used as an antigen-specific therapy to prevent autoimmune disease. Transplantation of syngeneic HSCs and/or HPCs avoids the need for conditioning regimens in a host and represents a novel, safe and effective strategy for preventing or treating autoimmune disease conditions.

With respect to autoimmune diabetes, proinsulin is proposed to be the key autoantigen.

In accordance with the present invention, therefore, syngeneic transplantation of HSCs and/or HPCs encoding proinsulin enables proinsulin expression in resting APCs and this results in the prevention of the development of autoimmune diabetes. This is a safe and effective antigen-specific strategy applicable to autoimmune diabetes as well as other autoimmune conditions.

Accordingly, the present invention contemplates a method for preventing or otherwise reducing the risk of development and/or reducing the severity of an autoimmune-mediated condition in an animal or avian species. The method involves collecting HSCs and/or HPCs either from the animal or avian species to be treated or from a compatible donor, genetically modifying some or all of the HSCs and/or HPCs such that they express genetic material encoding one or more autoantigens associated with the particular autoimmune disease and introducing these into the animal or avian species to be treated. Presentation of the autoantigen by APCs is proposed to induce T cell unresponsiveness or tolerance and/or protective immunity. The HSCs and/or HPCs may be collected from bone marrow or isolated from peripheral blood, cord blood or other convenient sites such as the liver. Once genetically modified, the cells are generally infused into the subject such that they enter the peripheral blood. This route of administration includes infusion or introduction to the liver such as via the portal vein.

In one embodiment, therefore, the present invention contemplates a method for generating an antigen presenting cell (APC) which presents an autoantigen associated with an autoimmune disease, the method comprising collecting a sample of hemopoetic stem cells (HSCs) and/or hemopoetic progenitor cells (HPCs) from a subject, introducing into one or more HSCs and/or HPCs genetic material encoding the autoantigen under conditions wherein the genetic material is expressed so that the HSCs and/or HPCs produce the autoantigens.

In a preferred embodiment, the autoimmune disease or condition is type 1 diabetes. The present invention extends, however, to a range of autoimmune diseases. With respect to autoimmune diabetes, the preferred autoantigen is proinsulin or an antigenic fragment or portion thereof.

The most preferred animal is a human but the present invention extends to other primates as well as livestock animals, laboratory test animals, companion animals, captured wild animals and avian species such as caged (aviary) birds, poultry birds and game birds.

The present invention provides kits in multiple compartmental form, the kit comprising a first compartment adapted to receive a source of HSCs and/or HPCs from a subject; a second compartment adapted to contain genetic material encoding an autoantigen; optionally a third or more compartments adapted to contain reagents wherein the kit comprises instructions for use comprising in a method comprising collecting a sample of hemopoetic stem cells (HSCs) and/or hemopoetic progenitor cells (HPCs) from a subject, introducing into one or more HSCs and/or HPCs genetic material encoding the autoantigen under conditions wherein the genetic material is expressed so that the HSCs and/or HPCs produce the autoantigens.

A list of abbreviations used herein is provided in Table 1.

TABLE 1
Abbreviations

| ABBREVIATION | DESCRIPTION |
|---|---|
| APC | antigen presenting cell |
| BM | bone marrow |
| BMT | bone marrow transplantation |
| CD | cluster differentiation antigens |
| DC | dendritic cells |
| F2.5 | FCS (2.5% v/v) |
| FCS | fetal calf serum |
| G | GM-CSF |
| G + T | mixture of GM-CSF and TGFβ1 |
| GM-CSF | granulocyte macrophage colony stimulating factor |
| HPC | hemopoietic progenitor cell |
| HSC | hemopoietic stem cell |
| i.v. | intravenous |
| iDC | immature dendritic cells |
| IL-10 | interleukin 10 |
| MHC | major histocompatibility complex |
| MLR | mixed lymphocyte reaction |
| NOD mice | non-obese diabetic mice |
| NOD-PI mice | NOD-transgenic proinsulin mouse |
| OVA | ovalbumin |
| PBL | peripheral blood leukocytes |
| PI | proinsulin |
| s.c. | subcutaneous |
| SD | standard deviation |
| T | TGFβ1 |
| TGFβ1 | transforming growth factor β1 |
| TID | type 1 diabetes |

### BRIEF DESCRIPTION OF THE FIGURES

FIG. 1 is a graphical representation showing that transplantation of NOD-PI BM inhibits diabetes development. (A) Diabetes incidence was significantly reduced in recipients of NOD-PI BM (▾) compared to NOD BM (▴) (P=0.003) or untreated NOD mice (▪) (P=0.001). NOD BM recipients did not differ from untreated controls. Data are pooled from two experiments in which BMT from NOD-PI or NOD mice was performed in parallel. (B) Diabetes incidence was significantly reduced in recipients of T cell-depleted NOD-PI BM (▾) compared to T cell-depleted NOD BM (▴) (P=0.003) or untreated NOD mice (▪) (P=0.036). NOD BM recipients did not differ from untreated controls. Data are pooled from three experiments in which BMT from NOD-PI or NOD mice was performed in parallel.

FIG. 2 presents micrographic and graphical representations showing that transplantation of T cell-depleted NOD-PI BM prevents insulitis but not sialitis. (A) Islets free of inflammatory infiltrate (insulitis) were common in recipients of NOD-PI BM and infiltration, when present, was restricted to the periphery of islets (arrow). (B) Extensively infiltrated islets (*) were common in recipients of NOD BM. (C) Insulitis was significantly reduced in recipients of T cell-depleted NOD-PI BM (▴) compared to NOD BM (▾) cells (P=0.008). Data for age-matched NOD (▪) and NOD-PI (♦) mice are included for comparison. (D) The number of sublingual gland inflammatory foci (sialitis score) did not differ between BMT and untreated mice. Individual mouse scores are pooled from two experiments in which BMT from NOD and NOD-PI mice was performed in parallel (horizontal bar indicates mean).

FIG. 3 presents graphical representations showing that transfer of NOD-PI hematopoietic stem cells (HSC) or hematopoietic progenitor cells (HPC) prevents diabetes development. (A) diabetes incidence was significantly reduced in recipients of NOD-PI HSC (▾) compared to recipients of NOD HSC (▴) (P=0.019) or untreated NOD mice (▪) (P=0.029). NOD HSC recipients and untreated NOD mice did not differ significantly. (B) Diabetes incidence was significantly reduced in recipients of NOD-PI HPC (▾) compared to recipients of NOD HPC (▴) (P=0.035) or untreated NOD mice (▪) (P=0.021). NOD HPC recipients and untreated NOD mice did not differ significantly. Data are from one (A) or two (B) experiments in which BMT from NOD-PI and NOD mice was performed in parallel.

FIG. 4 provides a graphical representation and tabulated data showing reconstitution of peripheral blood leucocytes (PBL) in recipients of T cell-depleted NOD or NOD-PI BM. (A) PBL were markedly depleted at 10-14 days but reconstituted by 8 weeks after irradiation and BMT in both NOD (▾) and NOD-PI (▴) T cell-depleted BM recipients, compared to untreated age-matched NOD mice (▪). (B) PBL subsets in recipients of T cell-depleted BM from NOD or NOD-PI donors reconstituted similarly and were similar to age-matched NOD controls. Data are mean±SD from two experiments in which BMT from NOD and NOD-PI mice were performed in parallel.

FIG. 5 is a graphical representation showing T cell recall responses to ovalbumin (OVA) immunization. Mice were immunised subcutaneously with OVA 100 days post-BMT and recall responses of splenic T cells measured 14 days later. T cell proliferation in the presence of OVA was similar for age-matched control NOD mice (▪) and recipients of T cell-depleted NOD (▾) or NOD-PI (▴) BM. Open symbols indicate proliferation in the absence of OVA. Data are from individual mice pooled from three separate experiments in which NOD and NOD-PI BMT were performed in parallel.

FIG. 6 is a graphical representation of BM cultured in GM-CSF/IL-4 or GM-CSF/TGF-β1, cells harvested at day 5 and cell surface markers analysed by flow cytometry. Numbers denote the percentage of cells falling in that quadrant (A). GM-CSF/IL-4 or GM-CSF/TGF-β1 cultured BM was harvested at day 5 and endocytic activity measured by uptake of FITC-dextran. Plots show FITC-dextran uptake vs CD86 expression for CD11c-gated cells from GM-CSF/IL-4 cultured BM or FITC-dextran uptake vs CD11c expression for bulk GM-CSF/TGF-β1 cultured BM (B). BM was cultured in GM-CSF/TGF-β1 and cells harvested at day 5. Cell surface markers expressed on Gr-1⁺ and CD 1c⁺ cells were analysed using 4-colour flow cytometry. Upper left dot plot shows gating used for analysis of Gr-1⁺-gated and CD11c⁺-gated cells. Histogram overlays show Gr-1-gated (shaded) and CD11c-gated (open) cells (C). iDC from G+T BM expressed low levels of MHC class II and co-stimulation molecules and were weak stimulators in the mixed lymphocyte reaction (MLR).

FIG. 7 is a graphical representation showing that G+T BM from NOD-PI, but not control NOD mice, significantly inhibited (p<0.01) diabetes development when transferred i.v. to 4 week-old female NOD mice.

FIG. 8 is a series of flow cytometric dot blots revealing that G+T BM contained large numbers of undifferentiated CD11c⁻/CD11b⁺/Gr-1⁺ myeloid cells, in addition to CD11c⁺/CD11b⁺/Gr-1⁻ iDC.

FIG. 9 is a graphical representation showing proinsulin-encoding Gr-1⁺ cells inhibits diabetes treated at four weeks.

FIG. 10 is a graphical representation showing proinsulin-encoding Gr-1⁺ cells inhibits diabetes treated at four weeks (1.8×10⁶ CD11c-depleted i.v.).

FIG. 11 is a photographic representation of Gr-1+ myeloid cells differentiate to CD11c+/MHC class II+ DC in vivo. Gr-1⁺ cells were purified from GM-CSF/TGF-β1-cultured proinsulin-NOD BM by depletion of CD11c+ cells, CFSE-labelled and injected directly into the spleen. Frozen sections of spleen were stained for immunofluorescence analysis. Localisation of CFSE- and antibody-labelled cells was performed by immunofluorescence microscopy. Panels show CFSE labelled cells (left), cells visualised with texas red conjugated mAb (centre) and merged images (right).

### DETAILED DESCRIPTION OF THE INVENTION

The present invention provides a safe and effective protocol for treating and/or preventing autoimmune disease conditions.

The protocol generally involves the steps of:—
(i) collecting a sample of HSCs and/or HPCs from an subject;
(ii) genetically modifying all or some of the HSCs and/or HPCs so that the HSCs and/or HPCs produce one or more autoantigens associated with the autoimmune disease; and
(iii) introducing the genetically modified HSCs and/or HPCs into the same subject or a compatible subject and which then eventually become APCs expressing the autoantigen.

The above steps may be combined and/or the order changed. Additional steps may also be included.

Reference to a “subject” such as a human subject as well as an animal or avian subject. The terms “individual” and “subject” in relation to the animal being treated may be used interchangeably. An “animal” includes a human, primate, livestock animal (e.g. sheep, horse, cow, horse, donkey, goat, pig), laboratory test animal (e.g. rabbit, mouse, rat, guinea pig), companion animal (e.g. dog, cat) or captured wild animal. An “avian species” includes caged or aviary birds, poultry birds (e.g. chickens, bantams, geese, turkeys) and game birds.

The most preferred animal in terms of medical science is a human. The present invention extends, however, to veterinary uses of the protocol to reduce autoimmune disease conditions in non-human animals.

The HSCs and/or HPCs are generally obtained from a sample of bone marrow such as from drilling into the hip bone. However, the present invention further extends to isolating and where necessary sorting HSCs and HPCs from peripheral blood including cord blood and blood from the liver. The cells are generally introduced into the recipient via, for example, i.v. injection or infusion into the peripheral blood system or liver via the portal vein. However, direct introduction into a recipient's bone marrow, although not preferred, is nevertheless contemplated by the present invention.

The process of the present invention may be “syngeneic”, “allogeneic” or “xenogeneic” with respect to the subjects within an animal species from which HSCs and/or HPCs are isolated and the subjects who receive the cells. A “syngeneic” process means that the subject from which the HSCs and/or HPCs are derived has the same MHC genotype as the recipient of the genetically modified HSCs and/or HPCs. An “allogeneic” process is where the HSCs and/or HPCs are from a MHC-incompatible subject to the subject to which the HSCs and/or HPCs are to be introduced. A “xenogeneic” process is where the HSCs and/or HPCs are from a different species to that to which the HSCs and/or HPCs are introduced. Preferably, the method of the present invention is conducted as a syngeneic process. To the extent that either an allogeneic or xenogeneic process is utilized, it should be understood that it may be necessary to modify the protocol such that any immunological responses, which may occur due to the mixing of foreign immuno-competent cells, are minimised.

Accordingly, the present invention contemplates a method for preventing or otherwise minimizing the risk of development of or reducing the severity of an autoimmune condition in a subject, said method comprising introducing into said subject, HSCs and/or HPCs which have been genetically modified such that they now produce one or more autoantigens associated with the autoimmune condition.

More particularly, the present invention provides a method for generating an antigen presenting cell (APC) which presents an autoantigen associated with an autoimmune disease, the method comprising collecting a sample of hemopoetic stem cells (HSCs) and/or hemopoetic progenitor cells (HPCs) from a subject, introducing into one or more HSCs and/or HPCs genetic material encoding the autoantigen under conditions wherein the genetic material is expressed so that the HSCs and/or HPCs produce the autoantigens.

The HSCs and/or HPCs are developed into APCs expressing particular autoantigens. Examples of APCs include but are not limited to dendritic cells, B-lymphocytes, epithelial cells or macrophages.

As indicated above, the subject includes a human, non-human animal and avian subject. Preferably, the subject is a human. The subject (e.g. human) may have pre-clinical diabetes or may be at risk of developing diabetes or may have clinical diabetes.

Also as indicated above, the method may involve the syngeneic, allogeneic or xenogeneic administration of HSCs and/or HPCs to a subject. However, a syngeneic protocol is preferred.

Accordingly, in a preferred embodiment, the present invention provides a method for preventing or otherwise minimizing the risk of developing or reducing the severity of an autoimmune disease in a subject, said method comprising introducing into said subject syngeneic HSCs and/or HPCs which have been genetically modified to produce one or more autoantigens associated with the autoimmune condition.

The preferred autoimmune disease is autoimmune diabetes, also known as type 1 diabetes or insulin-dependent diabetes. However, the present invention extends to the use of the subject protocol in the treatment of a range of autoimmune conditions. The only criterion is that an autoantigen associated with the disease condition be known. Examples of autoimmune conditions contemplated herein include inter alia systemic lupus, Crohn's disease, cardiomyopathy, hemolytic anemia, fibromyalgia, Graves' disease, ulcerative colitis, vasculitis, multiple sclerosis, myasthenia gravis, myositis, neutropenia, psoriasis, chronic fatigue syndrome, juvenile arthritis, juvenile diabetes, scleroderma, psoriatic arthritis, Sjogren's syndrome, rheumatic fever, rheumatoid arthritis, scarcoidosis, idiopathic thrombocytopenic purpura (ITP), Hashimoto's disease, mixed connective tissue disease, interstitial cystitis, pernicious anemia, leukoencephalitis, alopecia greata, ankylosing spondylitis, primary biliary cirrhosis, anti-GBM nephritis, anti-TBM nephritis, anti-phospholipid syndrome, polymyalgia rheumatica, polymyositis, autoimmune Addison's disease, chronic active hepatitis, vitiligo, autoimmune hyperlipidemia, autoimmune myocarditis, temporal arteritis, autoimmune thyroid disease, axonal and neuronal neuropathies, Behçet's disease, bullous pemphigoid, allergic asthma, osteoarthritis, Chagas' disease, uveitis, chronic inflammatory demyelinating polyneuropathy (CIDP), cicatricial pemphigoid/benign mucosal pemphigoid, Cogan's syndrome, congenital heart block, Coxsackie myocarditis, demyelinating neuropathies, dermatomyositis, discoid lupus, phacoantigenic uveitis, polyarteritis nodosa, Dressler's syndrome, essential mixed cryoglobulinemia, Evan's syndrome, Goodpasture's syndrome, allergic rhinitis, Guillain-Barré syndrome, hypoganmmaglobulinemia, inclusion body myositis, vesiculobullous dermatosis, Wegener's granulomatisis, Meniere's disease, Lambert-Eaton syndrome, Mooren's ulcer, non-typical celiac disease, ocular cicatricial pemphigoid, pemphigus vulgaris, perivenous encephalomyelitis, post-pericardiotomy syndrome, scleritis, sperm and testicular autoimmunity, Stiff man's syndrome, subacute bacterial endocarditis (SBE), sympathetic ophthalmia, transverse myelitis and necrotizing myelopathy, type 1 autoimmune polyglandular syndrome, type 1I autoimmune polyglandular syndrome, pernicious anaemia and endometriosis.

According to a preferred embodiment, the present invention contemplates a method of preventing, minimizing the risk of development of or the severity of autoimmune diabetes in a human subject, said method comprising administering to said human subject an effective amount of HSCs and/or HPCs isolated from said human subject or from a syngeneic subject and which HSCs and/or HPCs have been genetically modified such that they express an autoantigen associated with autoimmune diabetes.

The preferred autoantigen is proinsulin or an immunogenic homolog or antigen derivative, part, fragment or portion thereof. The proinsulin is generally of human origin although humanized proinsulin molecules from, for example, pigs, sheep, horses, goats, mice or rats are also contemplated.

According to a most preferred embodiment, the present invention provides a method of preventing, minimizing the risk of development of or the severity of autoimmune diabetes in a human subject, said method comprising administering to said human subject an effective amount of HSCs and/or HPCs isolated from said human subject or from a syngeneic subject and which HSCs and/or HPCs have been genetically modified such that they produce proinsulin.

It is proposed herein that syngeneic transplantation of gene-modified HSC and/or HPCs is a novel approach to antigen-specific immunotherapy which advances the principle of regulating autoimmune disease from within the hematopoietic compartment.

Preferably, the autoimmune disease is diabetes and the autoantigen is proinsulin since proinsulin contains T cell epitopes implicated in human Rudy et al., Mol. Med. 1: 625-633, 1995) and mouse (Chen et al., J. Immunol. 167: 4926-4935, 2001) type 1 diabetes. In work leading up to the present invention, the inventors observed that NOD mice transgenically-expressing proinsulin targeted to APCs by an MHC class II promoter (French et al., 1997, supra) contained bone marrow which could be used to adoptively transfer protection against the development of autoimmune diabetes following bone marrow transplantation to a wild-type NOD mouse. Protection from diabetes was profound in recipients of bone marrow from NOD-PI mice. By transferring highly purified HSC or HPCs, protection can be attributed to the APC progeny of engrafted HSCs/HPCs rather than other potentially immunoregulatory cells transferred in whole or T cell-depleted bone marrow. Most importantly, transfer of small numbers of genetically-modified HSCs totally prevents diabetes. HSC transplantation also demonstrates that diabetes transferred by wild-type NOD HSC is due to generation of diabetogenic T cells de novo rather than to transfer of diabetogenic T cells. Nevertheless, diabetogenic T cells clearly either do not develop or fail to acquire effector function in mice destined to express proinsulin in APC. These results provide proof of principle for genetically-modified HSCs as a therapeutic tool for autoimmune disease prevention.

The context in which antigen presentation occurs controls the balance of T cell immunity (Garza et al., J. Exp. Med. 191: 2021-2027, 2000; Frazer et al., J Immunool 167:6180-6187, 2001), Antigen presented by resting APC induces inactivation of T cells (Niimi et al., 1998, supra; Finkelman et al., 1996; supra; Hawiger et al., 2001, supra) and inhibits antigen-specific Ab production (Finkelman et al., 1996, supra). By transgenically targeting antigen expression, dendritic cells (DC) have been shown to play a key role in thymic deletion of antigen-specific T cells (Brocker et al., J. Exp. Med. 185: 541-550, 1997). Importantly, suppression of T cell responses in the periphery has also been described following administration of DC-targeted antigen (Finkelman et al., 1996, supra; Hawiger et al., 2001, supra). The ability to harness peripheral mechanisms of immune tolerance is likely to be the key to autoantigen-specific immunotherapy in subjects with autoreactive T cells.

While the inventors used myeloablative conditioning with irradiation to favour maximum engraftment of donor HSCs, this would not be acceptable in asymptomatic humans with pre-clinical autoimmune diabetes. However, as no MHC barrier exists, the approach is adaptable to protocols which require no toxic pre-bone marrow transplantation conditioning. By using HSCs derived from transgenic mice, the need to genetically-engineer HSCs ex vivo, which has been a major hurdle for HSC therapy, is by-passed. For human application, vectors capable of effectively transducing HSCs for long-term gene expression after engraftment are required. Gene expression can be effectively targeted to MHC class II⁺ APC in vivo by lentiviral-vector transduction of human HSC (Cui et al., Blood 99: 399-408, 2002). Therefore, a strategy whereby HSCs and/or HPCs are harvested from peripheral blood, optionally following cytokine-induced mobilization, genetically modified and reinfused is the preferred approach to the therapy of autoimmune disease.

Accordingly, another aspect of the present invention contemplates a method for treating or reducing the risk of development of or reducing the severity of diabetes in a human, said method comprising:—
(i) isolating HSCs and/or HPCs from peripheral blood or bone marrow, optionally including the steps of cytokine-mediated mobilization of the HSCs and/or HPCs;
(ii) genetically modifying the HSCs and/or HPCs so that the cells now produce proinsulin or an immunogenic homolog, antigenic derivative, part, fragment or portion thereof and continue to do so as APCs; and
(iii) infusing or introducing the genetically modified cells into a human subject.

Reference to genetically modifying HSCs and/or HPCs includes introducing nucleic acid molecules encoding proinsulin or other autoantigens into the genome of the cells. Generally, the nucleic acid molecule is DNA. The DNA may encode a full length autoantigen, multiple full length autoantigens or one or more fragments of one or more autoantigens which carry antigenic epitopes.

Yet another aspect of the present invention provides a vector useful for introducing genetic material encoding an autoantigen such as proinsulin, said vector comprising a nucleotide sequence encoding the autoantigen or an antigenic fragment thereof and a selectable marker.

A selectable marker in the vector allows for selection of targeted cells that have stably incorporated the autoantigen-encoding DNA. This is especially useful when employing relatively low efficiency transformation techniques such as electroporation, calcium phosphate precipitation and liposome fusion where typically fewer than 1 in 1000 cells will have stably incorporated the exogenous DNA. Using high efficiency methods, such as viral vectors and microinjection into nuclei, typically from 5-25% of the cells will have incorporated the DNA; and it is, therefore, feasible to screen the targeted cells directly without the necessity of first selecting for stable integration of a selectable marker. Either isogenic or non-isogenic DNA may be employed.

Examples of selectable markers include genes conferring resistance to compounds such as antibiotics, genes conferring the ability to grow on selected substrates, genes encoding proteins that produce detectable signals such as luminescence. A wide variety of such markers are known and available, including, for example, antibiotic resistance genes such as the neomycin resistance gene (neo) and the hygromycin resistance gene (hyg). Selectable markers also include genes conferring the ability to grow on certain media substrates such as the tk gene (thymidine kinase) or the hprt gene (hypoxanthine phosphoribosyltransferase) which confer the ability to grow on HAT medium (hypoxanthine, aminopterin and thymidine); and the bacterial gpt gene (guanine/xanthine phosphoribosyltransferase) which allows growth on MAX medium (mycophenolic acid, adenine and xanthine). Other selectable markers for use in mammalian cells and plasmids carrying a variety of selectable markers are described in Sambrook et al., Molecular Cloning—A Laboratory Manual, Cold Spring Harbour, N.Y., USA, 1990.

The selectable marker may depend on its own promoter for expression and the marker gene may be derived from a very different organism than the organism being targeted (e.g. prokaryotic marker genes used in targeting mammalian cells). However, it is preferable to replace the original promoter with transcriptional machinery known to function in the recipient cells. A large number of transcriptional initiation regions are available for such purposes including, for example, metallothionein promoters, thymidine kinase promoters, β-actin promoters, immunoglobulin promoters, SV40 promoters and human cytomegalovirus promoters. A widely used example is the pSV2-neo plasmid which has the bacterial neomycin phosphotransferase gene under control of the SV40 early promoter and confers in mammalian cells resistance to G418 (an antibiotic related to neomycin). A number of other variations may be employed to enhance expression of the selectable markers in animal cells, such as the addition of a poly(A) sequence and the addition of synthetic translation initiation sequences. Both constitutive and inducible promoters may be used.

The DNA is preferably modified by homologous recombination. The target DNA can be in any organelle of the HSC or HPC including the nucleus and mitochondria and can be an intact gene, an exon or intron, a regulatory sequence or any region between genes.

Homologous DNA is a DNA sequence that is at least 70% identical with a reference DNA sequence. An indication that two sequences are homologous is that they will hybridize with each other under stringent conditions (Sambrook et al., 1990, supra).

The present invention also provides a kit in multiple compartmental form, the kit comprising a first compartment adapted to receive a source of HSCs and/or HPCs from a subject; a second compartment adapted to contain genetic material encoding an autoantigen; optionally a third or more compartments adapted to contain reagents wherein the kit comprises instructions for use comprising in a method comprising collecting a sample of hemopoetic stem cells (HSCs) and/or hemopoetic progenitor cells (HPCs) from a subject, introducing into one or more HSCs and/or HPCs genetic material encoding the autoantigen under conditions wherein the genetic material is expressed so that the HSCs and/or HPCs produce the autoantigens.

In a related aspect, the present invention further provides a pharmaceutical kit comprising reagents and/or compartments adapted for use in isolation of HSCs and/or HPCs from peripheral blood or bone marrow, their genetic manipulation to express DNA encoding proinsulin or an antigenic part thereof or another autoantigen associated with autoimmune diabetes and/or means to reintroduce the genetically modified cells to a subject, either to the peripheral blood system or to bone marrow.

The present invention is further described by the following non-limiting Examples.

### EXAMPLE 1

### General Methods

Mice

NOD (non-obese diabetic) and NOD.scid mice were bred under specific-pathogen free conditions. NOD mice transgenic for mouse proinsulin II (NOD-PI) under control of the MHC class II (I-Eα) promoter (French et al., Diabetes 46: 34-39, 1997) were used after breeding to homozygosity. As the incidence of spontaneous diabetes is highest in wild-type NOD females, only females were used as recipients and bone marrow (BM) donors.

Antibodies and Flow Cytometry

Flow cytometric analysis was performed as described (Steptoe et al., J. Immunol. 168: 5032-5041, 2002). The following mAbs were purified from tissue culture supernatants and then used in conjugation reactions: Antibodies directed against Gr-1 (Ly-6G; RB6-8C5), F4/80 (F4/80), CD11b (5C6 or M1/70), CD11c (N418), MHC class II (10.2.16 [I-A^{k,g7,r,f,s}]), MHC class I (M1/42), M-CSF R (AFS-98), CD40 (FGK-45), B220 (RA3-6B2) and CD86 (GL-1) Streptavidin (SA)-fluorochrome conjugates (SA-FITC, SA-phycoerythrin, SA-allophycocyanin, SA-texas red), mAb to CD4 (CT-CD4), CD8α (CT-CD8a) and FITC, PE and Tricolor streptavidin conjugates were from Caltag (Burlingame, Calif.). Monoclonal antibodies directed to anti-CD3 (145-2C11), SCA-1 (E13-161.7), CD40 (3/23), MAC-3 (M3/84), CD13 (R3-242), CD62-L (MEL-14), CD31 (MEC13.3), CD43 (S7), CD11a (2D7), CD49d (R1-2) were purchased from PharMingen (San Diego, Calif.). In addition, anti-mouse FIRE, anti-CD3 (KT3), c-kit (ACK-2) was used.

For analysis of PBL mice were bled by retro-orbital venous sinus puncture with a fine glass capillary tube. Blood was collected in Alsever's anticoagulant, erythrocytes lysed and leukocytes stained and analyzed by flow cytometry. Leukocyte number determined with a hemocytometer was calibrated according to blood volume obtained. To control for inter-experimental variation, three age-matched female NOD were included in each analysis. Spleens were pressed through stainless steel mesh and cells suspended in RPMI containing 10% v/v FCS.

BM Preparation and Transfer

Mice (8-12 weeks old) were euthanased and femurs and tibiae collected into cold mouse-tonicity phosphate buffered saline (PBS). BM was flushed with ice cold PBS containing 2.5% v/v FCS (F2.5) (Trace Scientific, Melbourne Australia) and erythrocytes removed by NH₄Cl/TRIS buffer lysis. BM was washed in F2.5 and collected by centrifugation. For T cell depletion, BM was resuspended in F2.5, incubated with anti-CD3 mAb (KT3, 5 μg/ml) for 30 minutes at 4° C., then washed in F2.5. Antibody-labeled cells were depleted with anti-rat IgG immunomagnetic beads (Dynabeads, Dynal Biotech, Carlton South, Victoria, Australia). For sorted HSC or HPC, lineage marker-positive cells were depleted by immunomagnetic beads with a mix of FITC-conjugated lineage-specific mAb (KT3, M1/70, RA3.6B2, RB6-8C5, TER-119) at predetermined optimal concentrations. Remaining cells were labelled with anti-c-kit-phycoerythrin. For HSC isolation, lineage-depleted cells were also co-stained with anti-SCA-1-biotin, washed and stained with streptavidin-Tricolor. Lin⁻/c-kit⁺/SCA-1⁺ (HSC) or lin⁻/c-kit⁺ (HPC) cells were collected by sterile sorting (FACSII, Becton Dickinson, San Diego, Calif.). Irradiated mice received a total of 950 cGy (Theratron ⁶⁰Co, Theratronics, Kanata, ON, Canada) as two equal doses 2-3 hours apart. Cells (10⁷ BM or T cell-depleted BM, unless stated otherwise) were suspended in PBS and injected i.p. in 250 μl or i.v. in 100 μl for HSC (10³) and HPC (2.5×10⁴), 1-3 hours after irradiation in the case of irradiated mice. Irradiated mice were maintained on neomycin-supplemented drinking water for 3 weeks post-BMT. Any mice showing signs of physical distress in the immediate post-BMT period were euthanased and excluded from analysis.

T Cell Recall Response

Mice were immunized s.c. in the flank with 100 μg ovalbumin (OVA) (Grade V, Sigma St Louis, Mo.) in Complete Freund's Adjuvant (Difco, Detroit, Mich.). Spleens collected 14 days later from euthanased mice were pressed through stainless steel mesh and cells suspended in RPMI medium (GIBCO, Rockville, Mass.) containing 10% v/v FCS (Trace Scientific, Melbourne Australia), 10⁻³ M sodium pyruvate, 10⁻⁴ M non-essential amino acids (GIBCO), 2×10⁻³ M glutamine, 5×10⁻⁵ M 2-mercaptoethanol (Sigma). Splenocytes were plated in triplicate (2.5×10⁵ cells/well, 200 μl, 96 well flat-bottom plates) in the absence or presence of OVA (100 μg/ml). Cells were harvested on day 4 onto glass filter mats. ³H-thymidine (1 μCi/well) was added during the final 18 hours of culture. Incorporated radioactivity reflecting cell proliferation was measured in a scintillation counter (Topcount, Packard, Groningen, The Netherlands) and results expressed as mean stimulation index (SI)±standard deviation.

Monitoring for Diabetes

Mice were urine tested for glucose weekly with Diastix test strips (Bayer, Pymble, NSW Australia). In glycosuric mice blood glucose was measured with a meter (Accu-Chek, Roche, Castle Hill, NSW, Australia). Mice were considered diabetic when two consecutive blood glucose readings were >12.0 mM. Mice were euthanased when diabetic or showing sign of physical distress.

Assessment of Insulinitis and Sialitis

Pancreata were removed from euthanased mice and placed in Bouin's fixative for 24 h and then transferred to 70% v/v ethanol. Fixed tissues were embedded in paraffin and H&E stained sections separated by 250-300 μm were prepared. Insulitis was scored in a masked fashion as described (Leiter, Proc. Natl. Acad. Sci. USA 79: 630-634, 1982). Sublingual glands were removed and prepared as for pancreata. The number of inflammatory foci present were counted and expressed as a mean per section.

Statistical Analysis

Comparison of Kaplan-Meier survival curves was performed using the log-rank test (GraphPad Prism, GraphPad Software Inc., San Diego, Calif.). Insulitis scores between BMT groups were compared by Student-t-test.

### EXAMPLE 2

### Transplantation of NOD-PI Bone Marrow Prevents Diabetes

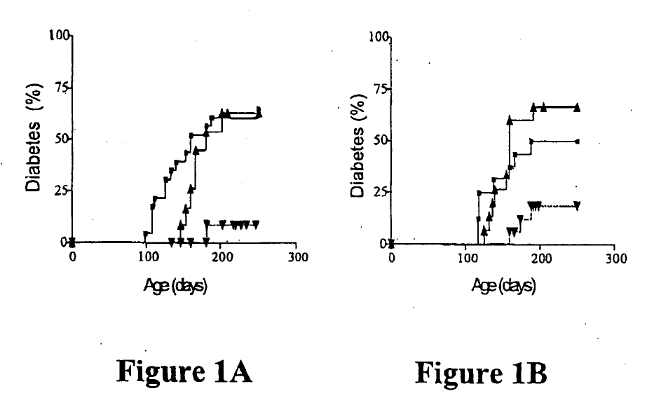

Whole BM from NOD or NOD-PI mice was transplanted to 4 week-old irradiated female NOD recipients. While the onset was delayed slightly, the overall incidence of diabetes in NOD BM recipients (7/12) was similar to untreated controls (15/23) (FIG. 1A). In contrast, diabetes was almost completely prevented in recipients of NOD-PI BM (1/16, P=0.0032) (FIG. 1A). NOD mice have an inherently high risk of thymoma development that is exacerbated by impaired immune surveillance or exposure to ionising radiation (Prochazka et al., Proc. Natl. Acad. Sci. USA 89: 3290-3294, 1992; Shultz et al., J. Immunol. 164: 2496-2507, 2000). Exclusion of mice diagnosed with thymomas at necropsy increased the proportion of mice with diabetes in both groups (NOD 7/10, NOD-PI 1/5) but the difference in diabetes incidence remained significant between groups (P±0.041). Because of their longer diabetes-free survival time, recipients of NOD-PI BM had a higher proportion of thymomas (11/16) compared to NOD BM recipients (2/12).

### EXAMPLE 3

### T Cell-Depletion does not Modify the Protective Effect of Transplanted NOD-PI BM

Separate studies had found that mature T cells in NOD BM were capable of transferring diabetes to immune-deficient NOD.scid mice. Diabetes development, following transfer of whole BM, might, therefore, have reflected the diabetogenic potential of transferred mature T cells. However, it was found that, whereas whole NOD BM transferred diabetes to at least 50% of non-irradiated T cell-deficient NOD.scid mice, no mice that received BM from NOD-PI mice developed diabetes.

As mature T cells in NOD, but not NOD-PI, BM could transfer diabetes, the effect of transplanting T cell-depleted BM to irradiated 4 week-old mice was tested. Recipients of T cell depleted NOD BM developed diabetes at a rate and incidence (10/15) similar to untreated controls (7/12) (FIG. 1B). In contrast, diabetes development was significantly less (3/17, P=0.003) in recipients of T cell-depleted NOD-PI BM (FIG. 1B). When mice with thymomas apparent at necropsy were removed from the analysis the difference between groups remained statistically significant (P=0.012).

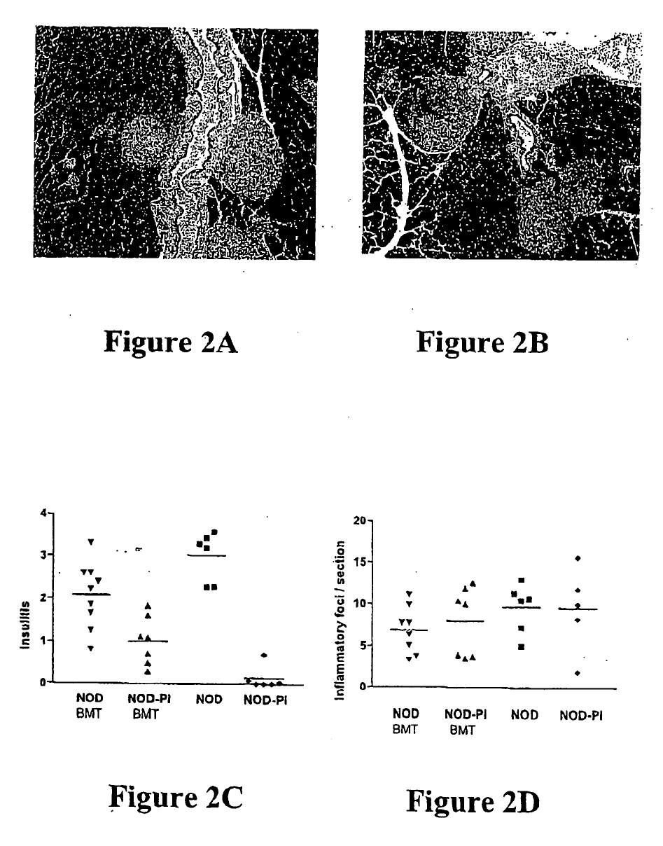

Cellular immune infiltration of pancreatic islets (insulitis) was assessed 100 days post-BMT (1×10⁷ T cell-depleted BM) In recipients of NOD-PI T cell-depleted BM, 54% of islets were free of insulitis (FIG. 2A) and mononuclear cell infiltration was restricted to the islet periphery (peri-insulitis). In contrast, in recipients of NOD T cell-depleted BM only 28% of islets were free of insulitis and there was extensive infiltration into the islets (FIG. 2B). These observations were reflected by a significantly reduced mean insulitis score for NOD-PI compared to NOD recipients (P=0.008) (FIG. 2C). Similar results were observed after transplantation of 5×10⁶ T cell-depleted BM. In contrast to insulitis, mononuclear cell infiltration of the sublingual gland (sialitis) was similar in recipients of either NOD-PI or NOD T cell-depleted BM and age-matched unmanipulated controls (FIG. 2D). This indicates that NOD-PI BM transfer protects specifically against islet auto-immunity.

### EXAMPLE 4

### PI-Encoding Hematopoietic Stem and Progenitor Cells Transfer Diabetes Prevention

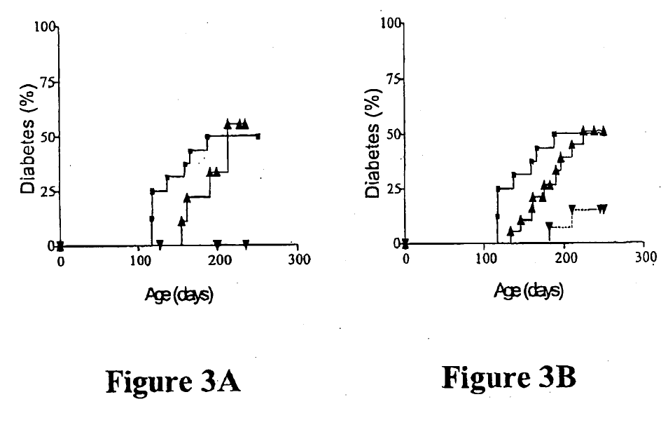

Hematopoietic stem cells (lin⁻/c-kit⁺/SCA-1⁺) or progenitor cells (HPC) (lin⁻/c-kit⁺) were sterile-purified from NOD and NOD-PI BM. To determine their effect on the development of diabetes, small numbers of either HSC or HPC were transplanted into irradiated 4 week-old recipients. Hematopoietic reconstitution was rapid and PBL populations were restored by 8 weeks post-BMT. Diabetes was totally prevented in recipients of NOD-PI HSC and its incidence significantly reduced in recipients of NOD-PI HPC (FIG. 3).

### EXAMPLE 5

### Diabetes Prevention by NOD-PI BM is not Due to Impaired Immune Reconstitution

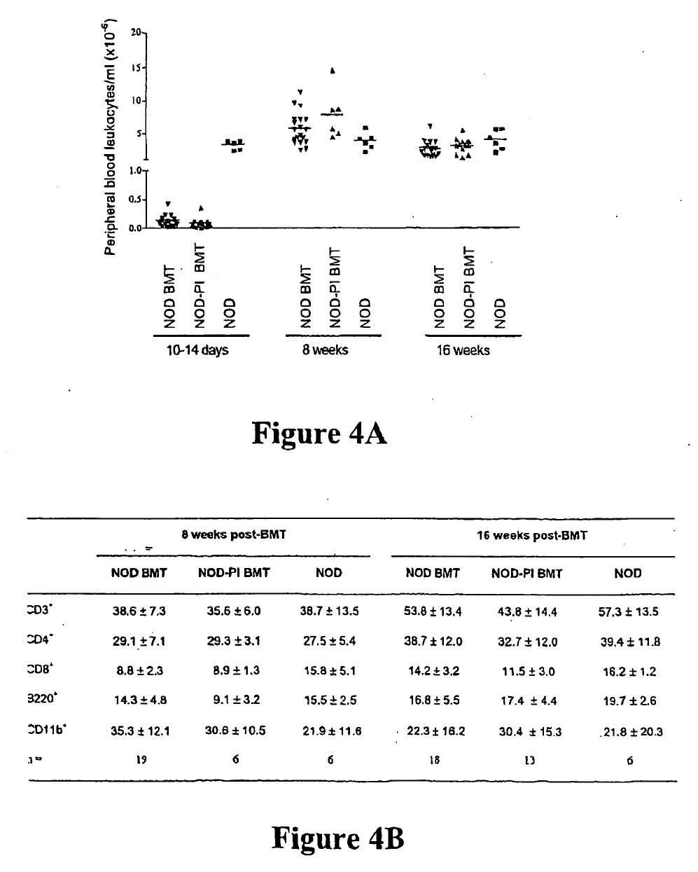

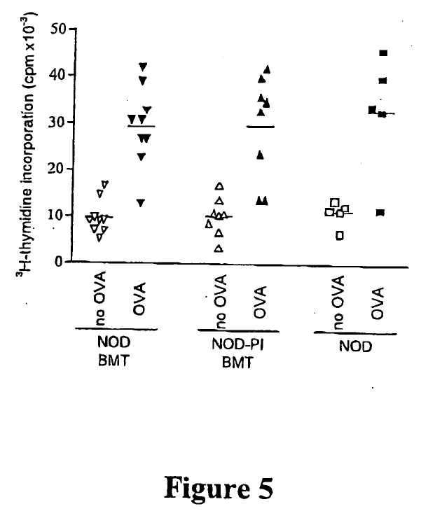

To exclude the possibility that the protective effect of NOD-PI BMT was the result of impaired immune reconstitution, peripheral blood leucocyte (PBL) populations were first analysed. Ten to fourteen days post-T cell-depleted BMT, circulating leucocytes were substantially reduced in number in both NOD and NOD-PI recipients (FIG. 4A). The proportions of T lymphocytes (CD4⁺, CD8⁺) and B lymphocytes (B220⁺) were reduced (50-75%, 25% and 80-85%, respectively) relative to age-matched controls, whereas the proportion of myeloid (CD11b+) cells was increased ˜2.5-fold. At 8 and 16 weeks post-BMT, total PBL count (FIG. 4A) and the relative proportion of PBL subsets (FIG. 4B) were normal, indicating similar reconstitution between groups. The ability of BMT recipients to mount a T cell-mediated immune response was then investigated. Normal age-matched NOD mice and recipients of NOD or NOD-PI T cell-depleted BM were immunized with ovalbumin (OVA) 100 days post-BMT. Two weeks later, in vitro recall responses to OVA were similar in untreated mice and either BMT group (FIG. 5). Thus, NOD-PI BMT was not associated with evidence of impaired immune reconstitution.

### EXAMPLE 6

### Cytokine-Stimulated Myeloid Cells Comprise Undifferentiated DC Precursors

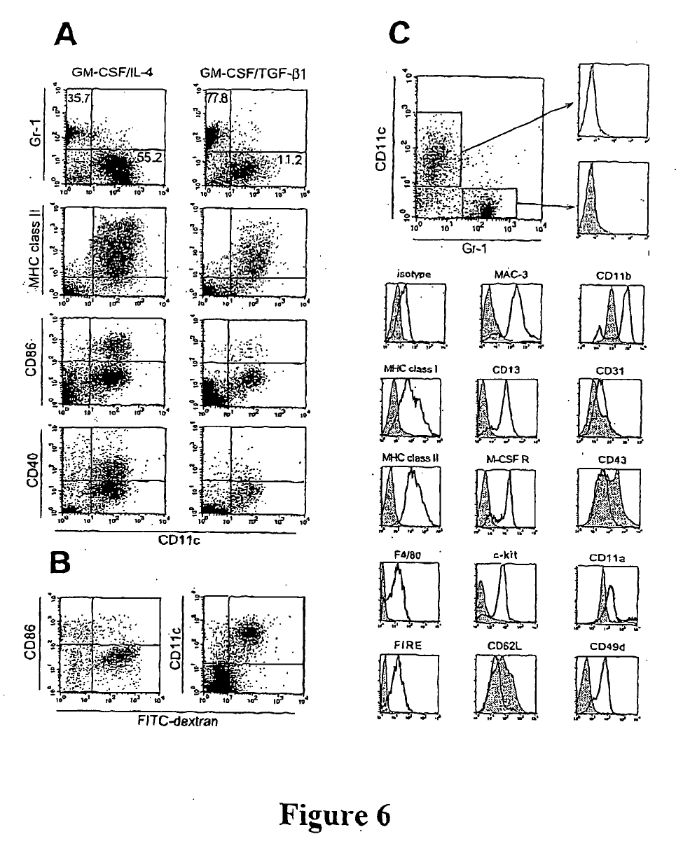

As a source of ‘immature’ DC (iDC), BM was cultured in GM-CSF and TGF-beta (G+T). These cultures contained mixtures of cell types, dominated by small round cells with annular or segmented nuclei that expressed the myeloid differentiation marker Gr-1, features characteristic with undifferentiated myeloid precursors. A small proportion of the cells had a monocyte-like or immature DC-like appearance and expressed low levels of MHC class II restricted primarily to intracellular granules. To further define these subpopulations of cells, BM cultured in comparisons were made between GM-CSF/TGF-β1 and GM-CSF/IL-4, as the latter contains a mix of pheotypically mature and immature DC along with small numbers of undifferentiated myeloid cells. In contrast to BM cells cultured in GM-CSF/IL-4, BM cultured in GM-CSF/TGF-β1 contained only a low frequency of cells expressing the DC-specific marker CD1 c (see FIG. 6A), the remainder comprising almost entirely Gr-1⁺ cells. Levels of antigen-presenting (MHC class II) and co-stimulation molecules (CD86 and CD40) expressed on CD11c+ DC in GM-CSF/TGF-βl-cultured BM were low and similar to those of phenotypically immature DC generated in GM-CSF/IL-4 (see FIG. 6A). Endocytic activity, a hallmark of functionally-immature (CD11c⁺/CD86^{lo})DC, were measured by FITC dextran uptake. In GM-CSF/IL-4 supplemeted cultures, only CD11c⁺/CD86^{lo} immature DC were edocytically active; in GM-CSF/TGF-β1-supplemented cultures, only CD11c⁺ cells were endocytically active.

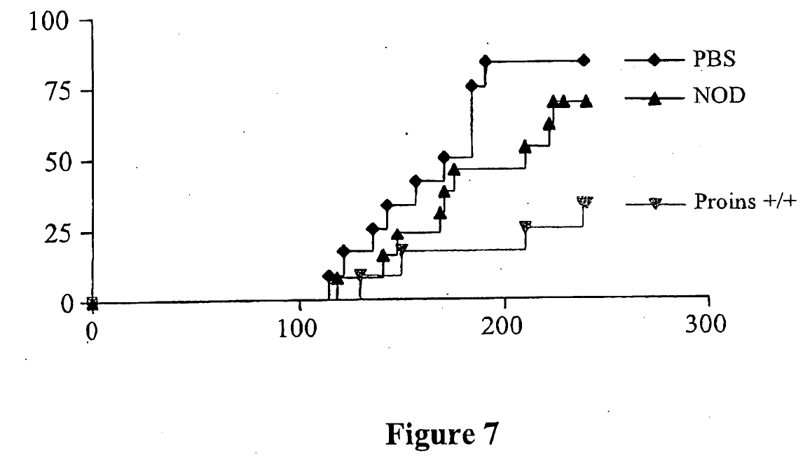

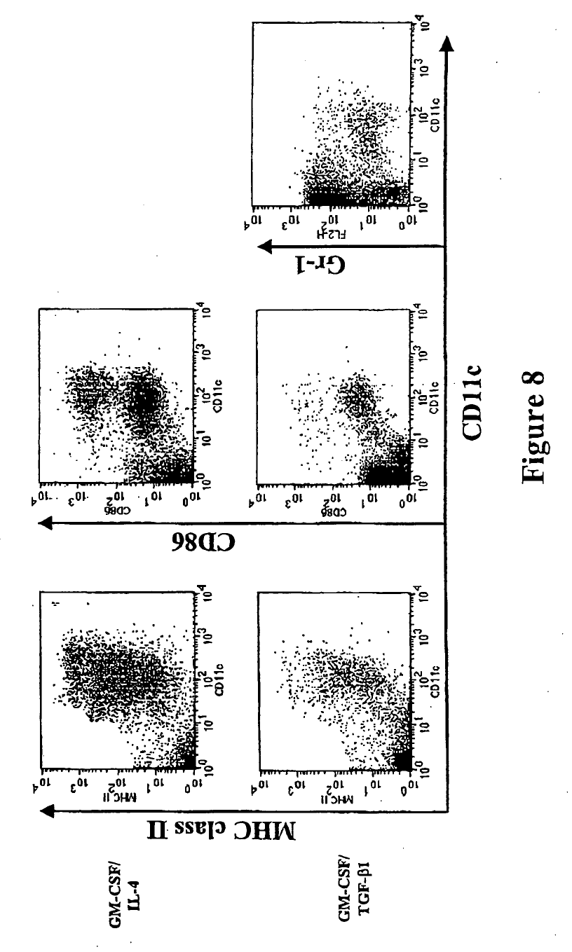

iDC from G+T BM expressed low levels of MHC class II and co-stimulation molecules (FIG. 6) and were weak stimulators in the mixed lymphocyte reaction. G+T BM from NOD-PI, but not control NOD mice, significantly inhibited (p<0.01) diabetes development when transferred i.v. to 4 week-old female NOD mice (FIG. 7). Further investigation revealed that G+T BM contained large numbers of undifferentiated CD11 c⁻/CD11b⁺/Gr-1⁺ myeloid cells in addition to CD11c⁺/CD11b⁺/Gr-1⁻ iDC (FIG. 8).

### EXAMPLE 7

### Precursor DC

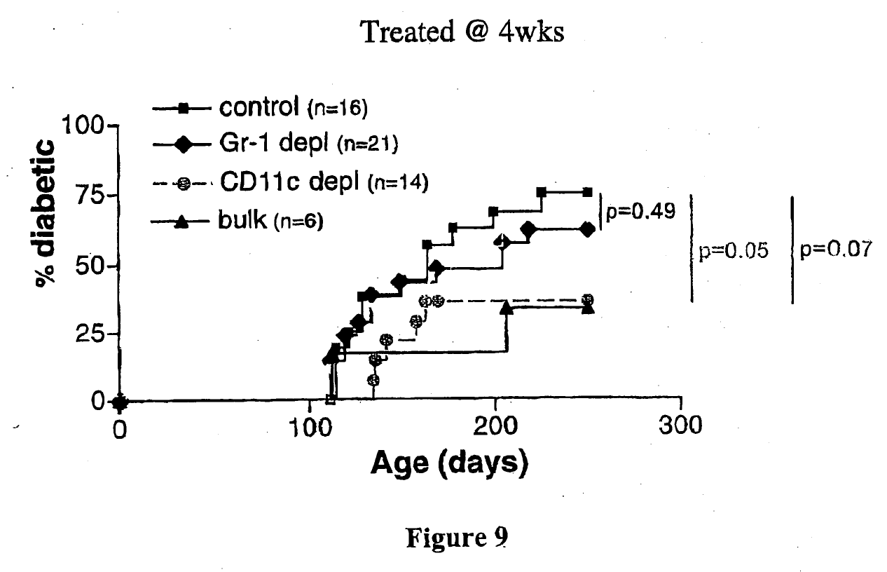

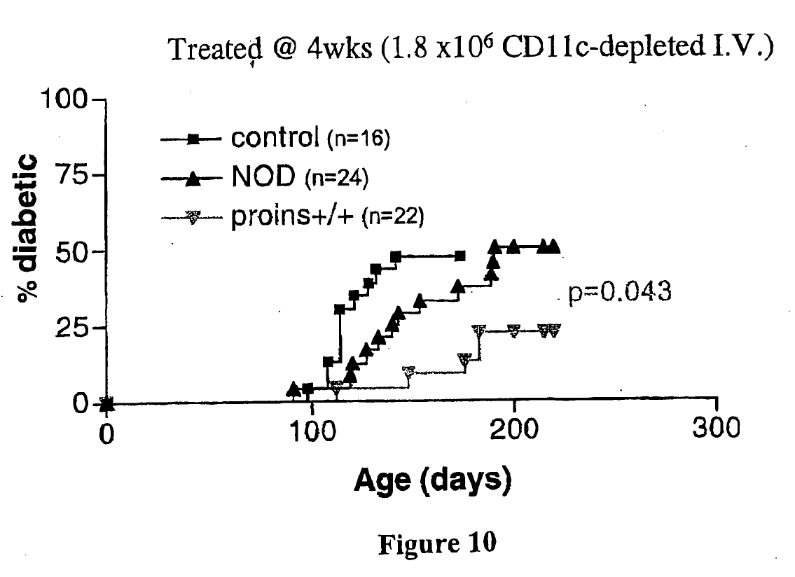

Depletion of Gr-1⁺ cells reduced the ability of G+T BM to inhibit diabetes, whereas depletion of CD11c⁺ iDC did not (FIG. 9). Transfer of purified Gr-1⁺ myeloid cells from NOD-PI (p<0.01) but not control NOD mice inhibited diabetes development in recipient mice, confirming the protective role of these cells (FIG. 10).

Unlike iDC, CD11c⁻/CD11b⁺/Gr-1⁺ myeloid cells did not rapidly acquire a mature CD11c⁺/CD86^{hi} phenotype 1n response to activational stimuli (LPS, anti-CD40). Instead, they gradually acquired mature DC characteristics over 5-7 days in culture in GM-CSF/IL 4/TNF-α. CD11c⁻/CD11b⁺/Gr-1⁺ cells present in G+T BM cultures therefore represent DC precursors. Hence, the foregoing data indicate that myeloid DC precursors encoding a disease-specific autoantigen (proinsulin) are able to prevent autoimmune disease.

### EXAMPLE 8

### Myeloid Cells Differentiate to CD11c+/MHC class II+ DC In Vivo

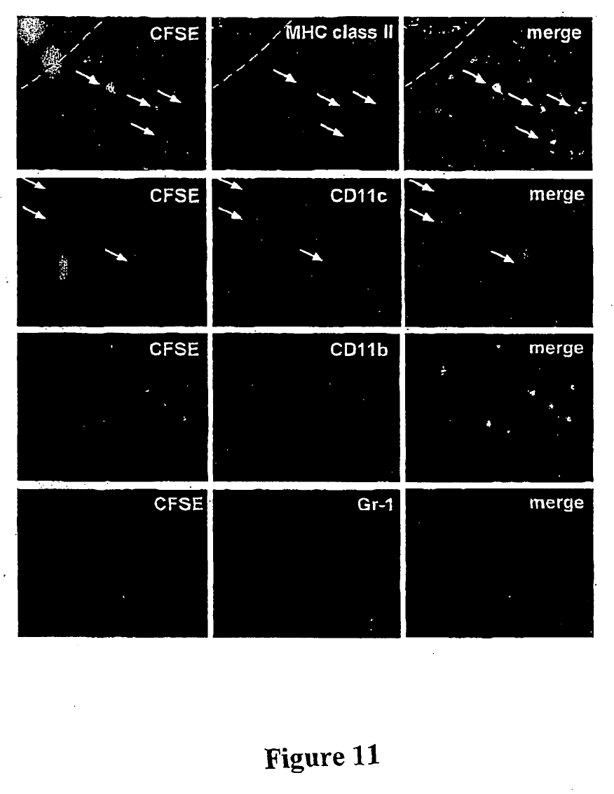

To determine the in vivo differentiation and survival of transferred cells, Gr1⁺ cells were purified from GM-CSF/TGF-β1-cultured proinsulin-NOD BM by depletion of CD11c+ cells. The cells were then CFSE labelled and injected directly into spleen. Frozen sections of spleen were stained for Immunofluorescence analysis. Localization of CFSE-and antibody labelled cells (either MHC class II, CD11c, CD11b or GR-1) was performed using Immunofluorescence microscopy. FIG. 11 demonstrates the identification of cell s which satin positive for CFSE and all four markers tested. The left panels show CFSE labelled cells, the middle panels shows cells visualized with texas red conjugated mAB, and the right panel shows merged images. Dual stating is indicated by the presence of bright white spots in the right panel.

Immunohistology

Cryostat sections (5 um) were cut from frozen OCT-embedded (Tissue-Tek, Miles Inc. Elkhart, Ind.) tissues, air dried and fixed with cold 100% ethanol prior to immunostaining or mounting. Avidin/biotin binding sites were blocked using avidin/biotin blocking reagents (Vector, Burlingame, Calif.) and non-specific protein interactions blocked with 1% BSA. Biotinylated primary antibodies were applied at predetermined optimal concentrations for one hour at room temperature. After washing, streptavidin-HRP (Vector ABC-Elite, Vector, Burlingame, Calif.) or streptavidin-texas red was applied for a further hour. Immunoperoxidase slides were washed and staining developed with enzyme substrate (Vector Red, Vector, Burlingame, Calif.). Immunofluorescence slides were rinsed and mounted in anti-fade reagent (DAKO Corp., Carpinteria, Calif.).

Cytospins

Cytospins were prepared using a cytofuge (Shandon, Pittsburgh, Pa.). Cytospins were stained using Diff Quik (Lab Aids Pty Ltd, Narrabeen, NSW Australia) or by immunohistochemistry as described.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Brocker, T., Riedinger, M., Karjalainen, K. 1997. Targeted expression of major histocompatibility complex (MHC) class II molecules demonstrates that dendritic cells can induce negative but not positive selection of thymocytes in vivo. J. Exp. Med. 185: 541-550.
Burt, R. K, Slavin, S., Burns, W. H., Marmont, A. M. 2002. Induction of tolerance in autoimmune diseases by hematopoietic stem cell transplantation: getting closer to a cure? Blood. 99: 768-784.
Castro-Malaspina, H., Harris, R. E., Gajewski, J., Ramsay, N., Collins, R., Dharan, B., King, R., Deeg, H. J. 2002. Unrelated donor marrow transplantation for myelodysplastic syndromes: outcome analysis in 510 transplants facilitated by the National Marrow Donor Program. Blood 99:1943-1951.
Chen, W., Bergerot, I., Elliott, J. F., Harrison, L. C., Abiru, N., Eisenbarth, G. S., Delovitch, T. L. 2001. Evidence that a peptide spanning the B-C junction of proinsulin is an early autoantigen epitope in the pathogenesis of type 1 diabetes. J. Immunol. 167: 4926-4935.
Cui, y., Golob, J., Kelleher, E., Ye, Z., Pardoll, D., Cheng, L. 2002. Targeting transgene expression to antigen-presenting cells derived from lentivirus-transduced human hematopoietic stem/progenitor cells. Blood 99: 399-408.
El-Badri, N. S., Wang, B. Y., Steele, A., Cherry, Marikar, Y., Mizobe, K., Good, R. A. 2000. Successful prevention of autoimmune disease by transplantation of adequate number of fully allogeneic hematopoietic stem cells. Transplantation 70: 870-877.
Frazer, I. H., De Kluyver, R., Leggatt, G. R., Guo, H. Y., Dunn, L., White, O., Harris, C., Liem, A., Lambert, P. 2001. Tolerance or immunity to a tumor antigen expressed in somatic cells can be determined by systemic proinflammatory signals at the time of first antigen exposure. J. Immunol. 167: 6180-6187.
French, M. B., Allison, J., Cram, D. S., Thomas, H. E., Dempsey-Collier, M., Silva, A., Georgiou, H. M., Kay, T. W., Harrison, L. C., Lew, A. M. 1997. Transgenic expression of mouse proinsulin II prevents diabetes in non-obese diabetic mice. Diabetes. 46: 34-39.
Garza, K. M., Chan, S. M., Suri, R., Nguyen, L. T., Odermatt, B., Schoenberger, S. P., Ohashi, P. S. 2000. Role of antigen-presenting cells in mediating tolerance and autoimmunity. J. Exp. Med. 191: 2021-2027.
Himeno, K., Good, R. A. 1988. Marrow transplantation from tolerant donors to treat and prevent autoimmune diseases in BXSB mice. Proc. Natl. Acad. Sci. U.S.A. 85: 2235-2239.
Ikehara, S., Ohtsuki, H., Good, R. A., Asamoto, H., Nakamura, T., Sekita, K., Muso, E., Tochino, Y., Ida, T., Kuzuya, H. et al. 1985. Prevention of type 1 diabetes in non-obese diabetic mice by allogeneic bone marrow transplantation. Proc. Natl. Acad. Sci. U.S.A. 82: 7743-7747.
Kaufman, C. L., Li, H., Ildstad, S. T. 1997. Patterns of hemopoietic reconstitution in non-obese diabetic mice: dichotomy of allogeneic resistance versus competitive advantage of disease-resistant marrow. J. Immunol. 158: 2435-2442.
Kirzner, R. P., Engelman, R. W., Mizutani, H., Specter, S., Good, R. A. 2000. Prevention of coronary vascular disease by transplantation of T cell-depleted bone marrow and hematopoietic stem cell preparation in autoimmune-prone w/BF(1)mice. Biol. Blood Marrow Transplant. 6: 513-522.
LaFace, D. M., Peck, A. B. 1989. Reciprocal allogeneic bone marrow transplantation between NOD mice and diabetes-non-susceptible mice associated with transfer and prevention of autoimmune diabetes. Diabetes. 38: 894-901.
Leiter, E. H. 1982. Multiple low-dose streptozotocin-induced hyperglycemia and insulitis in C57BL mice: influence of inbred background, sex and thymus. Proc. Natl. Acad. Sci. U.S.A. 79: 630-634.
Li, H., Kaufman, C. L., Boggs, S. S., Johnson, P. C., Patrene, K. D., Ildstad, S. T. 1996. Mixed allogeneic chimerism induced by a sublethal approach prevents autoimmune diabetes and reverses insulitis in non-obese diabetic (NOD)mice. J. Immuno. 1156: 380-388.
Prochazka, M., Gaskins, H. R., Shultz, L. D., Leiter, E. H. 1992. The non-obese diabetic scid mouse: model for spontaneous thymomagenesis associated with immunodeficiency. Proc. Natl. Acad. Sci. U.S.A. 89: 3290-3294.
Ratanatharathorn, V., Ayash, L., Lazarus, H. M., Fu, J., Uberti, J. P. 2001. Chronic graft-versus-host disease: clinical manifestation and therapy. Bone Marrow Transplant. 28: 121-129.
Rudy, G., Stone, N., Harrison, L. C., Colman, P. G., McNair, P., Brusic, V., French, M. B., Honeyman, M. C., Tait, B., Lew, A. M. 1995. Similar peptides from two beta cell autoantigens, proinsulin and glutamic acid decarboxylase, stimulate T cells of individuals at risk for insulin-dependent diabetes. Mol. Med. 1: 625-633.
Sambrook et al., Molecular Cloning—A Laboratory Manual, Cold Spring Harbor, N.Y., USA, 1990.
Shultz, L. D., Lang, P. A., Christianson, S. W., Gott, B., Lyons, B., Umeda, S., Leiter, E., Hesselton, R., Wagar, E. J., Leif, J. H. et al. 2000. NOD/LtSz-Rag1null mice: an immunodeficient and radioresistant model for engraftment of human hematolymphoid cells, HIV infection, and adoptive transfer of NOD mouse diabetogenic T cells. J. Immunol. 164: 2496-2507.
Steptoe, R. J., Ritchie, J. M., Harrison, L. C. 2002. Increased generation of dendritic cells from myeloid progenitors in autoimmune-prone non-obese diabetic mice. J. Immunol. 168: 5032-5041.

1. A method of preventing or treating insulin-dependent diabetes in a subject comprising introducing into said subject an APC which presents pro-insulin associated with an autoimmune disease, said method comprising collecting a sample of hemopoetic stem cells (HSCs) and/or hemopoetic progenitor cells (HPCs) from said subject, introducing into one or more HSCs and/or HPCs genetic material encoding said pro-insulin or an immunogenic homolog, part, fragment or portion thereof under conditions wherein said genetic material is expressed so that the HSCs and/or HPCs produce said pro-insulin or an immunogenic homolog, part, fragment or portion thereof.

2. The method of claim 1, wherein said APC is selected from a dendritic cell, B-lymphocyte, epithelial cell, monocyte and macrophage.

3. The method of claim 2, wherein said APC is a dendritic cell.

4. The method of claim 1, wherein said subject is selected from the group consisting of a human, primate, sheep, horse, cow, donkey, pig, goat, rabbit, mouse, rat, guinea pig, dog, cat, bird, chicken, bantams, geese and turkeys.

5. The method of claim 1, wherein said subject is a human.

6. The method of claim 1, wherein said cell is derived from bone marrow from the hip bone, bone marrow, cord blood, blood from liver, blood from a tissue and PBMCs.

7. The method of claim 6, wherein said cell is derived from bone marrow from a hip bone.

8. The method of claim 1, wherein said proinsulin is of human origin.

9. The method of claim 1, wherein said proinsulin is a humanised proinsulin, wherein said proinsulin is derived from the group selected of pig, cow, sheep, horse, goat, mouse and rat.

10. A method for treating or preventing insulin-dependent diabetes in a subject comprising,
(a) collecting a sample of hemopoetic stem cells (HSCs) and/or hemopoetic progenitor cells (HPCs) from a subject;
(b) introducing into one or more HSCs and/or HPCs genetic material encoding pro-insulin or an immunogenic homolog, part, fragment or portion thereof under conditions wherein said genetic material is expressed so that the HSCs and/or HPCs produce said pro-insulin or an immunogenic homolog, part, fragment or portion thereof; and
(c) infusing or introducing said genetically modified cells into said subject.

11. The method of claim 10, wherein said HSCs and/or HPCs undergo cytokine mediated mobilisation.

12. The method of claim 10, wherein said subject is selected from the group consisting of human, primate, sheep, horse, cow, donkey, pig, goat, rabbit, mouse, rat, guinea pig, dog, cat, bird, chicken, bantams, geese and turkeys.

13. The method of claim 10, wherein said subject is a human.

14. The method of claim 10, wherein said HSCs and HPCs are derived from a source selected from bone marrow from the hipbone, bone marrow, cord blood, blood from liver, blood from a tissue and PBMCs.

15. The method of claim 14, wherein said HSCs and HPCs are derived from bone marrow from a hipbone.

16. The method of claim 10, wherein said proinsulin is of human origin.

17. The method of claim 10, wherein said proinsulin is a humanized proinsulin, wherein said proinsulin is derived from a source selected from the group consisting of pig, cow, sheep, horse, goat, mouse and rat.

18. Use of an APC which has been genetically modified to present pro-insulin or an immunogenic homolog, part, fragment or portion thereof associated with insulin-dependent diabetes in the manufacture of a medicament for the treatment of insulin-dependent diabetes

19. The use of claim 18, wherein said APC is selected from the group consisting of a dendritic cell, B-lymphocyte, epithelial cell, monocyte and macrophage.

20. The use of claim 18, wherein said APC is a dendritic cell.

21. The use of claim 18, wherein said HSCs and/or HPCs are derived from a source selected from the group consisting of a human, primate, sheep, horse, cow, donkey, pig, goat, mouse, rat, guinea pig, dog, cat, chicken, bantam hen, geese and turkey.

22. The use of claim 21, wherein said HCSs and/or HPCs are derived from a human.

23. The use of claim 18, wherein said HSCs and/or HPCs are derived from a source selected from the group consisting of bone marrow from hipbone, bone marrow, cord blood, blood from liver, blood from a tissue and PBMCs.

24. The use of claim 18, wherein said proinsulin is of human origin.

25. The use of claim 18, wherein said proinsulin is a humanized proinsulin, wherein said proinsulin is derived from a source selected from the group consisting of pig, cow, sheep, horse, goat, mouse and rat.
